(19) United States
(12) Patent Application Publication
(10) Pub. No.: US 2014/0171705 A1
(43) Pub. Date: Jun. 19, 2014

(54) PROCESS AND APPARATUS FOR RECOVERING PRODUCT

(71) Applicant: UOP LLC, Des Plaines, IL (US)

(72) Inventors: Christian D. Freet, South Elgin, IL (US); Todd M. Kruse, Oak Park, IL (US); Christopher P. Nicholas, Evanston, IL (US)

(73) Assignee: UOP LLC, Des Plaines, IL (US)

(21) Appl. No.: 13/714,548

(22) Filed: Dec. 14, 2012

(51) Int. Cl. C07C 7/00; C07C 2/06

(52) U.S. Cl. CPC C07C 7/005; C07C 2/06
USPC 585/315; 585/519; 585/802

(57) ABSTRACT

A process and apparatus are disclosed for recovering a product stream by fractionation perhaps with compression of a C₅⁻ hydrocarbon stream. The C₅⁻ hydrocarbon stream may be taken from an overhead of a fractionation column. Fractionation includes a depentanizer column followed by a depropanizer column for producing a C₄ and C₅ stream. The recovered product stream may be oligomerized to produce larger oligomers. Oligomers may be delivered to a cracking reactor which may produce the C₅⁻ hydrocarbon stream.

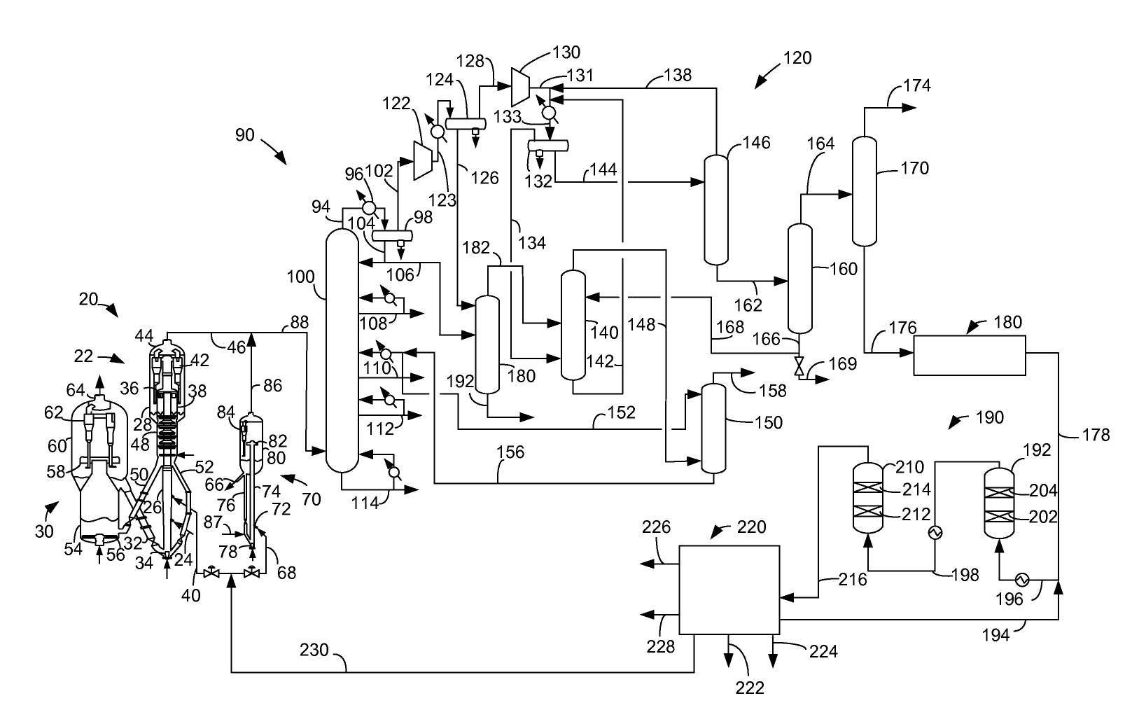

### FIELD OF THE INVENTION

The field of the invention is the recovery of hydrocarbon streams by separation and oligomerization of light olefins to heavier oligomers.

### BACKGROUND

The oligomerization of butenes is often associated with a desire to make a high yield of high quality gasoline product. What can be achieved when oligomerizing butenes can be limited. When oligomerizing butenes, dimerization is desired to obtain gasoline range material. However, trimerization and higher oligomerization can occur which can produce material heavier than gasoline such as diesel. Efforts to produce diesel by oligomerization have failed to provide high yields except through multiple passes.

When oligomerizing olefins from a fluid catalytic cracking (FCC) unit, there is often a desire to maintain a liquid phase within the oligomerization reactors. A liquid phase helps with catalyst stability by acting as a solvent to wash the catalyst of heavier species produced. In addition, the liquid phase provides a higher concentration of olefins to the catalyst surface to achieve a higher catalyst activity. Typically, this liquid phase in the reactor is maintained by hydrogenating some of the heavy olefinic product and recycling this paraffinic product to the reactor inlet.

To maximize propylene produced by the FCC unit, refiners may contemplate oligomerizing FCC olefins to make heavier oligomers and recycling heavier oligomers to the FCC unit. However, some heavy oligomers may be resistant to cracking down to propylene.

Improved apparatuses and processes are desired for recovering valuable products from product gases for use in an oligomerization zone.

### SUMMARY OF THE INVENTION

In the oligomerization of light olefins a hydrocarbon feed stream of C₄ and C₅ olefins is desired. Typically, a C₄ and C₅ olefin feed stream would be acquired by mixing a C₄ hydrocarbon stream from a bottoms stream of a C₃/C₄ splitter column with C₅ hydrocarbon stream from an overhead of a depentanizer column. However, the C₅ hydrocarbon stream is typically taken from a naphtha cut from a side of the FCC main column. This naphtha cut typically contains large concentrations of poisons such as mercaptans and thiophenes that can deactivate the oligomerization catalyst.

The apparatus and process may be used to recover cracked product or to produce hydrocarbon product which may include olefinic product. The apparatus and process are designed to recover a hydrocarbon stream comprising C₄ and C₅ olefins. A portion of a main column overhead stream or a compressed stream is fed as a depentanizer feed stream to a depentanizer column to provide a C₅-overhead stream which is then fed as a depropanizer feed stream to a depropanizer column to provide a stream comprising C₄ and C₅ hydrocarbons which may be recovered or used as an oligomerization feed to an oligomerization zone.

An object of the invention is the provision of a C₄ and C₅ olefinic feed stream with a lower concentration of poison.

### BRIEF DESCRIPTION OF THE DRAWING

The FIGURE is a schematic drawing of the present invention.

### DEFINITIONS

As used herein, the term “stream” can include various hydrocarbon molecules and other substances. Moreover, the term “stream comprising Cₓ hydrocarbons” or “stream comprising Cₓ olefins” can include a stream comprising hydrocarbon or olefin molecules, respectively, with “x” number of carbon atoms, suitably a stream with a majority of hydrocarbons or olefins, respectively, with “x” number of carbon atoms and preferably a stream with at least 75 wt-% hydrocarbons or olefin molecules, respectively, with “x” number of carbon atoms. Moreover, the term “stream comprising Cₓ⁺ hydrocarbons” or “stream comprising Cₓ⁺ olefins” can include a stream comprising a majority of hydrocarbon or olefin molecules, respectively, with more than or equal to “x” carbon atoms and suitably less than 10 wt-% and preferably less than 1 wt-% hydrocarbon or olefin molecules, respectively, with x−1 carbon atoms. Lastly, the term “Cₓ⁻ stream” can include a stream comprising a majority of hydrocarbon or olefin molecules, respectively, with less than or equal to “x” carbon atoms and suitably less than 10 wt-% and preferably less than 1 wt-% hydrocarbon or olefin molecules, respectively, with x+1 carbon atoms.

As used herein, the term “zone” can refer to an area including one or more equipment items and/or one or more sub-zones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “gasoline” can include hydrocarbons having a boiling point temperature in the range of about 25° to about 200° C. at atmospheric pressure.

As used herein, the term “diesel” or “distillate” can include hydrocarbons having a boiling point temperature in the range of about 150° to about 400° C. and preferably about 200° to about 400° C.

As used herein, the term “vacuum gas oil” (VGO) can include hydrocarbons having a boiling temperature in the range of from 343° to 552° C.

As used herein, the term “vapor” can mean a gas or a dispersion that may include or consist of one or more hydrocarbons.

As used herein, the term “overhead stream” can mean a stream withdrawn at or near a top of a vessel, such as a column.

As used herein, the term “bottom stream” can mean a stream withdrawn at or near a bottom of a vessel, such as a column.

As depicted, process flow lines in the FIGURE can be referred to interchangeably as, e.g., lines, pipes, feeds, gases, products, discharges, parts, portions, or streams.

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottom stream back to the bottom of the column. Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column.

As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, “taking a stream from” means that some or all of the original stream is taken.

The term “predominant” means a majority, suitably at least 80 wt-% and preferably at least 90 wt-%.

### DETAILED DESCRIPTION

The stream comprising C₄ and C₅ hydrocarbons may be obtained from a cracked product stream that may come from a fluid catalytic cracking (FCC) zone. The process and apparatus will be described as such, but the stream comprising C₄ and C₅ hydrocarbons may be obtained from other sources. In such an exemplary embodiment, the apparatus and process may be described with reference to four components shown in the FIGURE: an FCC zone 20, an FCC recovery zone 90, a pretreatment zone 180, an oligomerization zone 190, and an oligomerization recovery zone 220. Many configurations of the present invention are possible, but specific embodiments are presented herein by way of example. All other possible embodiments for carrying out the present invention are considered within the scope of the present invention.

The fluid catalytic cracking zone 20 may comprise a first FCC reactor 22, a regenerator vessel 30, and an optional second FCC reactor 70.

A conventional FCC feedstock and higher boiling hydrocarbon feedstock are a suitable FCC hydrocarbon feed 24 to the first FCC reactor. The most common of such conventional feedstocks is a VGO. Higher boiling hydrocarbon feedstocks to which this invention may be applied include a heavy bottom from crude oil, heavy bitumen crude oil, shale oil, tar sand extract, deasphalted residue, products from coal liquefaction, atmospheric and vacuum reduced crudes and mixtures thereof. The FCC feed 24 may include a recycle stream 230 to be described later.

The first FCC reactor 22 may include a first reactor riser 26 and a first reactor vessel 28. A regenerator catalyst pipe 32 delivers regenerated catalyst from the regenerator vessel 30 to the reactor riser 26. A fluidization medium such as steam from a distributor 34 urges a stream of regenerated catalyst upwardly through the first reactor riser 26. At least one feed distributor injects the first hydrocarbon feed in a first hydrocarbon feed line 24, preferably with an inert atomizing gas such as steam, across the flowing stream of catalyst particles to distribute hydrocarbon feed to the first reactor riser 26. Upon contacting the hydrocarbon feed with catalyst in the first reactor riser 26 the heavier hydrocarbon feed cracks to produce lighter gaseous cracked products while coke is deposited on the catalyst particles to produce spent catalyst.

The resulting mixture of gaseous product hydrocarbons and spent catalyst continues upwardly through the first reactor riser 26 and are received in the first reactor vessel 28 in which the spent catalyst and gaseous product are separated. Disengaging arms discharge the mixture of gas and catalyst from a top of the first reactor riser 26 through outlet ports 36 into a disengaging vessel 38 that effects partial separation of gases from the catalyst. A transport conduit carries the hydrocarbon vapors, stripping media and entrained catalyst to one or more cyclones 42 in the first reactor vessel 28 which separates spent catalyst from the hydrocarbon gaseous product stream. Gas conduits deliver separated hydrocarbon cracked gaseous streams from the cyclones 42 to a collection plenum 44 for passage of a cracked product stream to a first cracked product line 46 via an outlet nozzle and eventually into the FCC recovery zone 90 for product recovery.

Diplegs discharge catalyst from the cyclones 42 into a lower bed in the first reactor vessel 28. The catalyst with adsorbed or entrained hydrocarbons may eventually pass from the lower bed into a stripping section 48 across ports defined in a wall of the disengaging vessel 38. Catalyst separated in the disengaging vessel 38 may pass directly into the stripping section 48 via a bed. A fluidizing distributor delivers inert fluidizing gas, typically steam, to the stripping section 48. The stripping section 48 contains baffles or other equipment to promote contacting between a stripping gas and the catalyst. The stripped spent catalyst leaves the stripping section 48 of the disengaging vessel 38 of the first reactor vessel 28 stripped of hydrocarbons. A first portion of the spent catalyst, preferably stripped, leaves the disengaging vessel 38 of the first reactor vessel 28 through a spent catalyst conduit 50 and passes into the regenerator vessel 30. A second portion of the spent catalyst may be recirculated in recycle conduit 52 from the disengaging vessel 38 back to a base of the first riser 26 at a rate regulated by a slide valve to recontact the feed without undergoing regeneration.

The first riser 26 can operate at any suitable temperature, and typically operates at a temperature of about 150° to about 580° C. at the riser outlet 36. The pressure of the first riser is from about 69 to about 517 kPa (gauge) (10 to 75 psig) but typically less than about 275 kPa (gauge) (40 psig). The catalyst-to-oil ratio, based on the weight of catalyst and feed hydrocarbons entering the riser, may range up to 30:1 but is typically between about 4:1 and about 10:1. Steam may be passed into the first reactor riser 26 and first reactor vessel 28 at a rate between about 2 and about 7 wt-% for maximum gasoline production and about 10 to about 15 wt-% for maximum light olefin production. The average residence time of catalyst in the riser may be less than about 5 seconds.

The catalyst in the first reactor 22 can be a single catalyst or a mixture of different catalysts. Usually, the catalyst includes two catalysts, namely a first FCC catalyst, and a second FCC catalyst. Such a catalyst mixture is disclosed in, e.g., U.S. Pat. No. 7,312,370 B2. Generally, the first FCC catalyst may include any of the well-known catalysts that are used in the art of FCC. Preferably, the first FCC catalyst includes a large pore zeolite, such as a Y-type zeolite, an active alumina material, a binder material, including either silica or alumina, and an inert filler such as kaolin.

Typically, the zeolites appropriate for the first FCC catalyst have a large average pore size, usually with openings of greater than about 0.7 nm in effective diameter defined by greater than about 10, and typically about 12, member rings. Suitable large pore zeolite components may include synthetic zeolites such as X and Y zeolites, mordenite and faujasite. A portion of the first FCC catalyst, such as the zeolite portion, can have any suitable amount of a rare earth metal or rare earth metal oxide.

The second FCC catalyst may include a medium or smaller pore zeolite catalyst, such as exemplified by at least one of ZSM-5, ZSM-11, ZSM-12, ZSM-23, ZSM-35, ZSM-38, ZSM-48, and other similar materials. Other suitable medium or smaller pore zeolites include ferrierite, and erionite. Preferably, the second component has the medium or smaller pore zeolite dispersed on a matrix including a binder material such as silica or alumina and an inert filler material such as kaolin. These catalysts may have a crystalline zeolite content of about 10 to about 50 wt-% or more, and a matrix material content of about 50 to about 90 wt-%. Catalysts containing at least about 40 wt-% crystalline zeolite material are typical, and those with greater crystalline zeolite content may be used. Generally, medium and smaller pore zeolites are characterized by having an effective pore opening diameter of less than or equal to about 0.7 nm and rings of about 10 or fewer members. Preferably, the second FCC catalyst component is an MFI zeolite having a silicon-to-aluminum molar ratio greater than about 15. In one exemplary embodiment, the silicon-to-aluminum molar ratio can be about 15 to about 35.

The total catalyst mixture in the first reactor 10 may contain about 1 to about 25 wt-% of the second FCC catalyst, including a medium to small pore crystalline zeolite, with greater than or equal to about 7 wt-% of the second FCC catalyst being preferred. When the second FCC catalyst contains about 40 wt-% crystalline zeolite with the balance being a binder material, an inert filler, such as kaolin, and optionally an active alumina component, the catalyst mixture may contain about 0.4 to about 10 wt-% of the medium to small pore crystalline zeolite with a preferred content of at least about 2.8 wt-%. The first FCC catalyst may comprise the balance of the catalyst composition. The high concentration of the medium or smaller pore zeolite as the second FCC catalyst of the catalyst mixture can improve selectivity to light olefins. In one exemplary embodiment, the second FCC catalyst can be a ZSM-5 zeolite and the catalyst mixture can include about 0.4 to about 10 wt-% ZSM-5 zeolite excluding any other components, such as binder and/or filler.

The regenerator vessel 30 is in downstream communication with the first reactor vessel 28. In the regenerator vessel 30, coke is combusted from the portion of spent catalyst delivered to the regenerator vessel 30 by contact with an oxygen-containing gas such as air to regenerate the catalyst. The spent catalyst conduit 50 feeds spent catalyst to the regenerator vessel 30. The spent catalyst from the first reactor vessel 28 usually contains carbon in an amount of from 0.2 to 2 wt-%, which is present in the form of coke. An oxygen-containing combustion gas, typically air, enters the lower chamber 54 of the regenerator vessel 30 through a conduit and is distributed by a distributor 56. As the combustion gas enters the lower chamber 54, it contacts spent catalyst entering from spent catalyst conduit 50 and lifts the catalyst at a superficial velocity of combustion gas in the lower chamber 54 of perhaps at least 1.1 m/s (3.5 ft/s) under fast fluidized flow conditions. In an embodiment, the lower chamber 54 may have a catalyst density of from 48 to 320 kg/m³ (3 to 20 lb/ft³) and a superficial gas velocity of 1.1 to 2.2 m/s (3.5 to 7 ft/s). The oxygen in the combustion gas contacts the spent catalyst and combusts carbonaceous deposits from the catalyst to at least partially regenerate the catalyst and generate flue gas.

The mixture of catalyst and combustion gas in the lower chamber 54 ascends through a frustoconical transition section to the transport, riser section of the lower chamber 54. The mixture of catalyst particles and flue gas is discharged from an upper portion of the riser section into the upper chamber 60. Substantially completely or partially regenerated catalyst may exit the top of the transport, riser section 58. Discharge is effected through a disengaging device 58 that separates a majority of the regenerated catalyst from the flue gas. The catalyst and gas exit downwardly from the disengaging device 58. The sudden loss of momentum and downward flow reversal cause a majority of the heavier catalyst to fall to the dense catalyst bed and the lighter flue gas and a minor portion of the catalyst still entrained therein to ascend upwardly in the upper chamber 60. Cyclones 62 further separate catalyst from ascending gas and deposits catalyst through dip legs into a dense catalyst bed. Flue gas exits the cyclones 62 through a gas conduit and collects in a plenum 64 for passage to an outlet nozzle of regenerator vessel 30. Catalyst densities in the dense catalyst bed are typically kept within a range of from about 640 to about 960 kg/m³ (40 to 60 lb/ft³).

The regenerator vessel 30 typically has a temperature of about 594° to about 704° C. (1100° to 1300° F.) in the lower chamber 54 and about 649° to about 760° C. (1200° to 1400° F.) in the upper chamber 60. Regenerated catalyst from dense catalyst bed is transported through regenerated catalyst pipe 32 from the regenerator vessel 30 back to the first reactor riser 26 through the control valve where it again contacts the first feed in line 24 as the FCC process continues. The first cracked product stream in the first cracked product line 46 from the first reactor 10, relatively free of catalyst particles and including the stripping fluid, exit the first reactor vessel 28 through an outlet nozzle. The first cracked products stream in the line 46 may be subjected to additional treatment to remove fine catalyst particles or to further prepare the stream prior to fractionation. The line 46 transfers the first cracked products stream to the FCC recovery zone 90, which is in downstream communication with the FCC zone 20. In an aspect, the line 88 may carry first cracked products to the FCC recovery zone 90 after mixing with second cracked products in line 86. The FCC recovery zone 90 typically includes a main fractionation column 100 and a gas recovery section 120.

A recycle cracking stream in recycle cracking line 230 delivers an FCC recycle stream to the FCC zone 20. The recycle cracking stream is directed into a first FCC recycle line 40 with the control valve thereon opened. In an aspect, the recycle cracking stream may be directed into an optional second FCC recycle line 68 with the control valve thereon opened. The first FCC recycle line 40 delivers the first FCC recycle stream to the first FCC reactor 22 in an aspect to the riser 26 at an elevation above the first hydrocarbon feed in line 24. The second FCC recycle line 68 delivers the second FCC recycle stream to the second FCC reactor 70. Typically, both control valves on lines 40 and 68, respectively, will not be opened at the same time, so the recycle cracking stream goes through only one of the first FCC recycle line 40 and the second FCC recycle line 68. However, feed through both is contemplated.

The optional second FCC recycle stream may be fed to the optional second FCC reactor 70 in the second FCC recycle line 68 via feed distributor 72. The second FCC reactor 70 may include a second riser 74. The second FCC recycle stream is contacted with catalyst delivered to the second riser 74 by a catalyst return pipe 76 to produce cracked upgraded products. The catalyst may be fluidized by inert gas such as steam from distributor 78. Generally, the second FCC reactor 70 may operate under conditions to convert the second FCC recycle stream to second cracked products such as ethylene and propylene. A second reactor vessel 80 is in downstream communication with the second riser 74 for receiving second cracked products and catalyst from the second riser. The mixture of gaseous, second cracked product hydrocarbons and catalyst continues upwardly through the second reactor riser 74 and is received in the second reactor vessel 80 in which the catalyst and gaseous, second cracked products are separated. A pair of disengaging arms may tangentially and horizontally discharge the mixture of gas and catalyst from a top of the second reactor riser 74 through one or more outlet ports 82 (only one is shown) into the second reactor vessel 80 that effects partial separation of gases from the catalyst. The catalyst can drop to a dense catalyst bed within the second reactor vessel 80. Cyclones 84 in the second reactor vessel 80 may further separate catalyst from second cracked products. Afterwards, a second cracked product stream can be removed from the second reactor 84 through an outlet in a second cracked product line 86 in downstream communication with the second reactor riser 74. The second cracked product stream in line 86 is fed to the FCC recovery zone 90, in an aspect, optionally mixed with the first cracked product stream in line 88. The second cracked products may be fed to the FCC recovery zone 90 separately from the first cracked products. Separated catalyst may be recycled via a recycle catalyst pipe 76 from the second reactor vessel 80 regulated by a control valve back to the second reactor riser 74 to be contacted with the second FCC recycle stream.

In some embodiments, the second FCC reactor 70 can contain a mixture of the first and second FCC catalysts as described above for the first FCC reactor 22. In one preferred embodiment, the second FCC reactor 70 can contain less than about 20 wt-%, preferably less than about 5 wt-% of the first FCC catalyst and at least 20 wt-% of the second FCC catalyst. In another preferred embodiment, the second FCC reactor 70 can contain only the second FCC catalyst, preferably a ZSM-5 zeolite.

The second FCC reactor 70 may be in downstream communication with the regenerator vessel 30 and receive regenerated catalyst therefrom in line 87. In an embodiment, the first FCC reactor 22 and the second FCC reactor 70 both share the same regenerator vessel 30. Line 66 carries spent catalyst from the second reactor vessel 80 to the lower chamber 54 of the regenerator vessel 30. The catalyst regenerator is in downstream communication with the second FCC reactor 70 via line 66.

The same catalyst composition may be used in both reactors 22, 70. However, if a higher proportion of the second FCC catalyst of small to medium pore zeolite is desired in the second FCC reactor 70 than the first FCC catalyst of large pore zeolite, replacement catalyst added to the second FCC reactor 70 may comprise a higher proportion of the second FCC catalyst. Because the second FCC catalyst does not lose activity as quickly as the first FCC catalyst, less of the second catalyst inventory must be forwarded to the catalyst regenerator 30 in line 66 from the second reactor vessel 80, but more catalyst inventory may be recycled to the riser 74 in return conduit 76 without regeneration to maintain a high level of the second FCC catalyst in the second reactor 70.

The second reactor riser 74 can operate in any suitable condition, such as a temperature of about 425° to about 705° C., preferably a temperature of about 550° to about 600° C., and a pressure of about 140 to about 400 kPa, preferably a pressure of about 170 to about 250 kPa. Typically, the residence time of the second reactor riser 74 can be less than about 3 seconds and preferably is than about 1 second. Exemplary risers and operating conditions are disclosed in, e.g., US 2008/0035527 A1 and U.S. Pat. No. 7,261,807 B2.

The FCC recovery zone 90 comprises a main column 100. The main column 100 is a fractionation column with trays and/or packing positioned along its height for vapor and liquid to contact and reach equilibrium proportions at tray conditions and a series of pump-arounds to cool the contents of the main column. The main fractionation column is in downstream communication with the FCC zone 20 and can be operated with an top pressure of about 35 to about 172 kPa (gauge) (5 to 25 psig) and a bottom temperature of about 343° to about 399° C. (650° to 750° F.). The first cracked product stream and perhaps second cracked product stream in line 88 are directed to a lower section of an FCC main fractionation column 100. A variety of products are withdrawn from the main column 100. In this case, the main column 100 recovers an overhead stream of light products comprising unstabilized naphtha and lighter gases in a main overhead line 94. The overhead stream in the main overhead line 94 is condensed in a condenser and perhaps cooled in a cooler both represented by 96 before it enters a main column receiver 98 in downstream communication with the FCC zone 20 and the main overhead line 94 for separation. An overhead line 102 withdraws a light off-gas main receiver overhead stream of C₅ hydrocarbons, LPG and dry gas from the receiver 98. The main receiver overhead stream in the main receiver overhead line 102 should comprise at least 10 wt-% and preferably at least 15 wt-% C₅ hydrocarbons. An aqueous stream is removed from a boot in the receiver 98. A bottoms liquid stream of light unstabilized naphtha leaves the receiver 98 via an overhead receiver bottom line 104. A first portion of the bottoms liquid stream is directed back to an upper portion of the main column and a second portion in line 106 may be directed to a naphtha splitter column 180 in upstream communication with a gas recovery section 120. The main receiver overhead line 102 may feed the main receiver overhead stream to the gas recovery section 120.

Several other fractions may be separated and taken from the main column including an optional heavy naphtha stream in line 108, a light cycle oil (LCO) in line 110, a heavy cycle oil (HCO) stream in line 112, and heavy slurry oil from the bottom in line 114. Portions of any or all of lines 108-114 may be recovered while remaining portions may be cooled and pumped back around to the main column 100 to cool the main column typically at a higher entry location. The light unstabilized naphtha fraction preferably has an initial boiling point (IBP) temperature at or below the C₅ range; i.e., about 25° C. (77° F.) and preferably about 30° C. (86° F.), and an end point (EP) temperature at greater than or equal to about 127° C. (260° F.). The optional heavy naphtha fraction has an IBP temperature at or above about 127° C. (260° F.) and an EP temperature at or above about 200° C. (392° F.), preferably between about 204° and about 221° C. (400° and 430° F.), particularly at about 216° C. (420° F.). The heavy naphtha fraction will comprise the C₆ to C₁₂ hydrocarbon fraction. The LCO stream has an IBP temperature at or above about 127° C. (260° F.) if no heavy naphtha cut is taken or at about the EP temperature of the heavy naphtha if a heavy naphtha cut is taken and an EP in a range of about 260° to about 371° C. (500° to 700° F.) and preferably about 288° C. (550° F.). The HCO stream has an IBP temperature of the EP temperature of the LCO stream and an EP temperature in a range of about 371° to about 427° C. (700° to 800° F.), and preferably about 399° C. (750° F.). The heavy slurry oil stream has an IBP temperature of the EP temperature of the HCO stream and includes everything boiling at a higher temperature.

In the gas recovery section 120, the naphtha splitter column 180 may be located upstream of a primary absorber column 140 to improve the efficiency of the gas recovery unit. This embodiment has the advantage of decreasing the molecular weight of the naphtha fed to the gas recovery section 120. Therefore, the lean oil from the primary absorber bottom results in lower reboiling temperatures and also makes it possible to recover heat more efficiently. The gas recovery section 120 is shown to be an absorption based system, but any vapor recovery system may be used including a cold box system.

To obtain sufficient separation of light gas components the gaseous overhead receiver stream in overhead receiver line 102 may be a first compressor feed stream taken from the main overhead stream in main overhead line 94. The first compressor feed is compressed in a first compressor 122, also known as a wet gas compressor, which is in downstream communication with the main fractionation column 100, the main column overhead receiver 98 and the overhead line 102 of the main overhead receiver. Any number of compressor stages may be used, but typically dual stage compression is utilized. In dual stage compression, a compressed steam in compressor effluent line 123 from the first compressor 122 is cooled and enters a first compressor receiver 124 in downstream communication with the first compressor 122 to be separated between liquid and vapor.

If one compression stage is used, a liquid compressor receiver bottom stream in a first compressor receiver bottom line 126 from a bottom of the compressor receiver 124 is fed as stripper column feed to the stripper column 146, which arrangement is not shown in the FIGURE. If two compression stages are used, as shown in the FIGURE, liquid in line 126 from a bottom of the compressor receiver 124 and the unstabilized naphtha in line 106 from a bottom line 104 of the main fractionation column overhead receiver 98 flow into a naphtha splitter 180 in downstream communication with the compressor receiver 124. In an embodiment, these streams may join and flow into the naphtha splitter 180 together. In an aspect shown in the FIGURE, line 126 flows into the naphtha splitter 180 at a higher elevation than line 106.

If one compressor stage is used, compressed gas in the overhead compressor receiver stream in overhead compressor receiver line 128 from a top of the compressor receiver 124 may enter a primary absorber column 140. If two compressor stages are used as shown in the FIGURE, compressed gas in the overhead compressor receiver stream in overhead compressor receiver line 128 enters a second compressor 130 as a second compressor feed stream taken from the overhead stream in main overhead line 94. The second compressor 130 is also known as a wet gas compressor and is in downstream communication with the first compressor receiver 124 and the main fractionation column 100. A compressed stream from the second compressor 130 in line 131 may be joined by streams in lines 138 and 142, and they are cooled and fed to a second compressor receiver 132 in downstream communication with the second compressor 130 for separation. Compressed overhead gas from a top of the second compressor receiver 132 travels in an overhead line 134 to enter a primary absorber 140 at a lower point than an entry point for a naphtha splitter overhead stream in line 182. The primary absorber 140 may be in downstream communication with an overhead of the second compressor receiver 132. A liquid compressor receiver bottom stream comprising a stripper column feed from a bottom of the second compressor receiver 132 travels in compressor receiver bottom line 144 and is fed to a stripper column 146. The stripper column 146 is in downstream communication with the first compressor 122 and the second compressor 130 if there is one. In an aspect, the stripper column 146 is in downstream communication with the first compressor receiver bottom line 126 and/or the second compressor receiver bottom line 144 if there is one.

The first compression stage compresses gaseous fluids to a pressure of about 345 to about 1034 kPa (gauge) (50 to 150 psig) and preferably about 482 to about 690 kPa (gauge) (70 to 100 psig). The second compression stage compresses gaseous fluids to a pressure of about 1241 to about 2068 kPa (gauge) (180 to 300 psig).

The naphtha splitter column 180 may split a naphtha stream into a heavy naphtha bottoms, typically C₇⁺ hydrocarbons, in a bottom line 192 and a light naphtha overhead, typically C₇⁻ hydrocarbons, in an overhead line 182. The overhead stream from the naphtha splitter column 180 is carried in the overhead line 182 to the primary absorber column 140. Therefore, only light naphtha is circulated in the gas recovery section 120. The compressed overhead compressor receiver stream in line 134 may enter the primary absorber column 140 which is in downstream communication with the naphtha splitter column 180 via naphtha splitter overhead line 182. The naphtha splitter column 180 may be operated at a top pressure to keep the overhead in liquid phase, such as about 344 to about 3034 kPa (gauge) (50 to 150 psig) and a temperature of about 135° to about 191° C. (275° to 375° F.).

The gaseous hydrocarbon stream in lines 134 fed to the primary absorber column 140 is contacted with naphtha from the naphtha splitter overhead in line 182 to effect a separation between C₃⁺ and C₂⁻ hydrocarbons by absorption of the heavier hydrocarbons into the naphtha stream upon counter-current contact. A depentanized naphtha stream in line 168 taken from the bottom of a depentanizer column 160 to be described subsequently may be delivered to the primary absorber column 140 at a higher elevation than the naphtha splitter overhead stream in line 182 to effect further separation of C₃⁺ from C₂⁻ hydrocarbons. The primary absorber column 140 utilizes no condenser or reboiler but may have one or more pump-arounds to cool the materials in the column. The primary absorber column may be operated at a top pressure of about 1034 to about 2068 kPa (gauge) (150 to 300 psig) and a bottom temperature of about 27° to about 66° C. (80° to 150° F.). A predominantly liquid C₃⁺ stream with some amount of C₂⁻ material in solution in line 142 from the bottoms of the primary absorber column is returned to line 131 upstream of the condenser to be cooled and returned to the second compressor receiver 132.

An off-gas stream in line 148 comprising a predominantly C₂⁻ stream with some larger hydrocarbons from a top of the primary absorber 140 is directed to a lower end of a secondary or sponge absorber 150. A circulating stream of LCO in line 152 diverted from line 110 absorbs most of the remaining C₅⁺ material and some C₃-C₄ material in the off-gas stream in line 148 by counter-current contact. LCO from a bottom of the secondary absorber in line 156 richer in C₃⁺ material than the circulating stream in line 152 is returned in line 156 to the main column 100 via the pump-around for line 110. The secondary absorber column 150 may be operated at a top pressure just below the pressure of the primary absorber column 140 of about 965 to about 2000 kPa (gauge) (140 to 290 psig) and a bottom temperature of about 38° to about 66° C. (100° to 150° F.). The overhead of the secondary absorber 150 comprising dry gas of predominantly C₂⁻ hydrocarbons with hydrogen sulfide, amines and hydrogen is removed in line 158 and may be subjected to further separation to recover ethylene and hydrogen.

A stripper column feed comprising a compressor receiver bottom stream in the first compressor receiver bottom line 126 of the first compressor receiver 124 or the second compressor receiver bottom line 144 of the second compressor receiver 132 may be fed to the stripper column 146. Most of the C₂⁻ material is stripped from the C₃-C₇ material and removed in a stripper overhead stream of the stripper column 146 and returned to line 131 via stripper overhead line 138. The overhead gas in line 138 from the stripper column comprising C₂⁻ material and LPG and some light naphtha is returned to line 131 perhaps without first undergoing condensation. The condenser on line 131 will partially condense the stripper overhead stream from line 138 and the compressed stream in line 131 which are both mixed with the bottoms stream 142 from the primary absorber column 140 to provide a mixed, condensed stream in line 133. The mixed, condensed stream in line 133 will undergo vapor-liquid separation in the second compressor receiver 132. The stripper column 146 may be in downstream communication with the main fractionation column 100, the compressor 122 or 130 via a bottom line 126 or 144 of the respective compressor receiver 124 or 132, the FCC reactor zone 20, a bottom of the primary absorber 140 and an overhead of the naphtha splitter 180. The stripper column 146 may be run at a pressure above the compressor 130 discharge at about 1379 to about 2206 kPa (gauge) (200 to 320 psig) and a temperature of about 38° to about 149° C. (100° to 300° F.). The bottoms product of the stripper column 146 in line 162 may be rich in light naphtha.

Typically, gas recovery sections utilize a debutanizer column to debutanize the stripper bottoms stream in line 162 to provide a debutanized C₄-overhead stream which is then sent to a splitter column to separate C₃ from C₄ hydrocarbons. The splitter column bottoms product comprising C₄ hydrocarbons would have to be combined with a C₅ stream from a depentanizer overhead stream to provide a stream comprising C₄ and C₅ hydrocarbons. The depentanizer feed stream would typically come from a naphtha cut taken from a side of the main column 100 which would have additional poisons that may deactivate a downstream catalyst such as an oligomerization catalyst. The present invention instead takes a depentanizer feed stream from the overhead stream in the main overhead line 94 or from the main overhead receiver stream in the main overhead receiver line 102 from the main fractionation column 100 in one embodiment or from the compressed stream from the compressor 122 or 130 in another embodiment and depentanizes it in the depentanizer column 160. The depentanizer column 160 may be in downstream communication with the main overhead line 94 or the main overhead receiver line 102 from the main fractionation column 100 in one embodiment, the compressor 122 or 130 in separate embodiments or the bottom line 162 of the stripper column 146 in a further aspect. The depentanizer column 160 may also be in downstream communication with the FCC zone 20, the bottom of the primary absorber 140 and an overhead line 182 of the naphtha splitter 180.

The FIGURE shows that the liquid bottoms stream from the stripper column 146 comprising depentanizer column feed in stripper bottoms line 162 may be fed to a depentanizer column 160. The depentanizer column 160 separates the depentanizer feed into a vaporous depentanizer overhead stream comprising C₅⁻ hydrocarbons and a liquid depentanized bottoms stream comprising C₆⁺ hydrocarbons and no more than about 10 wt-% C₅ hydrocarbons. The depentanized bottoms stream in bottoms line 166 may be split between a recycle stream that may be recycled to the primary absorber in recycle line 168 and a product gasoline stream in line 169 through a control valve thereon. The primary absorber 140 is then in downstream communication with the depentanizer bottom line 166 via recycle line 168. The depentanized naphtha recycled to the primary absorber column 140 in recycle line 168 assists in the absorption of C₃⁺ materials. Typically, 25 to 50 wt-% of the depentanized naphtha is recycled to the primary absorber 140 in line 168 to control the recovery of light hydrocarbons. The depentanizer column may be operated at a top pressure of about 862 to about 1551 kPa (gauge) (125 to 225 psig) and a bottom temperature of about 149° to about 204° C. (300° to 400° F.). The pressure should be maintained as low as possible to maintain reboiler temperature as low as possible while still allowing complete condensation with typical cooling utilities without the need for refrigeration.

The depentanizer overhead stream in a depentanizer overhead line 164 from the depentanizer column 160 is condensed to provide a net depentanizer overhead stream comprising C₅-hydrocarbons which is taken as the depropanizer feed stream in the depentanizer overhead line 164. The depropanizer feed stream comprises no more than about 10 wt-% and, preferably, no more than about 5 wt-% C₆ hydrocarbons and at least about 5 wt-%, suitably at least about 10 wt-% and preferably at least about 20 wt-% C₅ hydrocarbons. The depropanizer feed stream is taken from the depentanizer column 160, in an aspect, the depentanizer overhead stream in the depentanizer overhead line 164, and is fed to a depropanizer column 170 which is in downstream communication with the overhead line 164 of the depentanizer column 160. In an aspect, the depropanizer column 170 is in direct communication and downstream communication with the depentanizer column 160 via the depentanizer overhead line 164.

In the depropanizer column 170, C₃ hydrocarbons are separated from C₄ and C₅ hydrocarbons. The depropanizer overhead stream comprising C₃ hydrocarbons in a depropanizer overhead line 174 may be recovered or further processed in a C₃ splitter to recover propylene product. A depropanized bottom stream comprising C₄ and C₅ hydrocarbons in the depropanized bottom line 176 may be recovered for blending in a gasoline pool as product. In an embodiment, the depropanized bottom stream can be taken as an oligomerization feed stream. The depropanizer column 170 may be operated with a top pressure of about 690 to about 1723 kPa (gauge) (100 to 250 psig) and a bottom temperature of about 38° to about 121° C. (100° to 250° F.).

Before the oligomerization feed stream can be fed to the oligomerization zone 190, the oligomerization feed stream in depropanized bottom line 176 may require purification. Many impurities in the oligomerization feed stream can poison an oligomerization catalyst. Carbon dioxide and ammonia can attack acid sites on the catalyst. Sulfur containing compounds, oxygenates, and nitriles can harm oligomerization catalyst. Acetylene and diolefins can polymerize and produce gums on the catalyst or equipment. Consequently, the oligomerization feed stream may be purified in an optional pretreatment zone 180. The pretreatment zone 180 may be in downstream communication with the depentanizer column 160 and the depropanizer column 170. The pretreatment zone 180 may include a mercaptan extraction unit to remove mecaptans, a selective hydrogenation reactor to minimize diolefins and acetylenes and/or a nitrile removal unit such as a water wash unit to reduce the concentration of oxygenates and nitriles in the oligomerization feed stream in line 176. A drier may follow the nitrile removal unit.

A pretreated oligomerization feed stream is provided in oligomerization feed stream line 178. The light olefin oligomerization feed stream in line 178 may be taken from the depropanizer column and particularly from a bottom line 176 of the depropanizer column 170. The oligomerization feed stream need not be obtained from a cracked stream but may come from another source. The oligomerization feed stream may comprise C₄ hydrocarbons such as butenes, i.e., C₄ olefins, and butanes. Butenes include normal butenes and isobutene. The oligomerization feed stream in oligomerization feed stream line 178 may comprise C₅ hydrocarbons such as pentenes, i.e., C₅ olefins, and pentanes. Pentenes include normal pentenes and isopentenes. Typically, the oligomerization feed stream will comprise about 20 to about 80 wt-% olefins and suitably about 40 to about 75 wt-% olefins. In an aspect, about 55 to about 75 wt-% of the olefins may be butenes and about 25 to about 45 wt-% of the olefins may be pentenes. Ten wt-%, suitably 20 wt-%, typically 25 wt-% and most typically 30 wt-% of the oligomerization feed may be C₅ olefins.

The oligomerization feed line 178 feeds the oligomerization feed stream to an oligomerization zone 190 comprising an oligomerization reactor 192 which may be in downstream communication with the FCC recovery zone 90, the depentanizer column 160, the depropanizer column 170 and the pretreatment zone 180. Specifically, oligomerization zone 190 comprising the oligomerization reactor 192 is in downstream communication with the bottoms line 176 of the depropanizer column 170 and the overhead line 164 of the depentanizer column 160. The oligomerization feed stream in oligomerization feed line 178 may be mixed with an oligomerate recycle stream in one or more recycle lines represented by recycle line 194 prior to entering the oligomerization zone 190 to provide an oligomerization feed stream in an oligomerization feed conduit 196.

The oligomerization zone 190 comprises a first oligomerization reactor 192. The first oligomerization reactor 192 may be preceded by an optional guard bed for removing catalyst poisons that is not shown. The first oligomerization reactor 192 contains the oligomerization catalyst. An oligomerization feed stream may be preheated before entering the first oligomerization reactor 192 in an oligomerization zone 190. The first oligomerization reactor 192 may contain a first catalyst bed 202 of oligomerization catalyst. The first oligomerization reactor 192 may be an upflow reactor to provide a uniform feed front through the catalyst bed, but other flow arrangements are contemplated. In an aspect, the first oligomerization reactor 192 may contain an additional bed or beds 204 of oligomerization catalyst. C₄ olefins in the oligomerization feed stream oligomerize over the oligomerization catalyst to provide an oligomerate stream comprising C₄ olefin dimers and trimers. C₅ olefins that may be present in the oligomerization feed stream oligomerize over the oligomerization catalyst to provide an oligomerate comprising C₅ olefin dimers and trimers and co-oligomerize with C₄ olefins to make C₉ olefins. The oligomerization produces other oligomers with additional carbon numbers.

In an aspect, the oligomerization zone 190 may include one or more additional oligomerization reactors 210. The oligomerization effluent from oligomerization reactor 190 may be heated and fed to the optional additional oligomerization reactor 210 in oligomerization effluent line 198. It is contemplated that the first oligomerization reactor 192 and the additional oligomerization reactor 210 may be operated in a swing bed fashion to take one reactor offline for maintenance or catalyst regeneration or replacement while the other reactor stays online. In an aspect, the additional oligomerization reactor 210 may contain a first bed 212 of oligomerization catalyst. The additional oligomerization reactor 210 may also be an upflow reactor to provide a uniform feed front through the catalyst bed, but other flow arrangements are contemplated. In an aspect, the additional oligomerization reactor 210 may contain an additional bed or beds 214 of oligomerization catalyst. Remaining C₄ olefins in the oligomerization feed stream oligomerize over the oligomerization catalyst to provide an oligomerate comprising C₄ olefin dimers and trimers. Remaining C₅ olefins, if present in the oligomerization feed stream, oligomerize over the oligomerization catalyst to provide an oligomerate comprising C₅ olefin dimers and trimers and co-oligomerize with C₄ olefins to make C₉ olefins. Over 90 wt-% of the C₄ olefins in the oligomerization feed stream can oligomerize in the oligomerization zone 190. Over 90 wt-% of the C₅ olefins in the oligomerization feed stream can oligomerize in the oligomerization zone 190. If more than one oligomerization reactor is used, conversion is achieved over all of the oligomerization reactors 192, 210 in the oligomerization zone 190.

An oligomerate conduit 216, in communication with the oligomerization reactor zone 190, withdraws an oligomerate stream from the oligomerization zone 190. The oligomerate conduit 216 may be in downstream communication with the first oligomerization reactor 192 and the additional oligomerization reactor 210.

The oligomerization zone 190 may contain an oligomerization catalyst. The oligomerization catalyst may comprise a zeolitic catalyst. The zeolite may comprise between 5 and 95 wt-% of the catalyst. Suitable zeolites include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. In a preferred aspect, the oligomerization catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the oligomerization catalyst comprises an MTT zeolite.

The oligomerization catalyst may be formed by combining the zeolite with a binder, and then forming the catalyst into pellets. The pellets may optionally be treated with a phosphoric reagent to create a zeolite having a phosphorous component between 0.5 and 15 wt-% of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present invention is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark Versal. A preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina.

A suitable oligomerization catalyst is prepared by mixing proportionate volumes of zeolite and alumina to achieve the desired zeolite-to-alumina ratio. In an embodiment, about 5 to about 80, typically about 10 to about 60, suitably about 15 to about 40 and preferably about 20 to about 30 wt-% MTT zeolite and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize surface acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm. However, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

In an embodiment, the oligomerization catalyst may be a solid phosphoric acid catalyst (SPA). The SPA catalyst refers to a solid catalyst that contains as a principal ingredient an acid of phosphorous such as ortho-, pyro- or tetraphosphoric acid. SPA catalyst is normally formed by mixing the acid of phosphorous with a siliceous solid carrier to form a wet paste. This paste may be calcined and then crushed to yield catalyst particles or the paste may be extruded or pelleted prior to calcining to produce more uniform catalyst particles. The carrier is preferably a naturally occurring porous silica-containing material such as kieselguhr, kaolin, infusorial earth and diatomaceous earth. A minor amount of various additives such as mineral talc, fuller's earth and iron compounds including iron oxide may be added to the carrier to increase its strength and hardness. The combination of the carrier and the additives preferably comprises about 15 to 30 wt-% of the catalyst, with the remainder being the phosphoric acid. The additive may comprise about 3 to 20 wt-% of the total carrier material. Variations from this composition such as a lower phosphoric acid content are possible. Further details as to the composition and production of SPA catalysts may be obtained from U.S. Pat. No. 3,050,472, U.S. Pat. No. 3,050,473 and U.S. Pat. No. 3,132,109 and from other references. If the oligomerization catalyst is SPA, the oligomerization feed stream in the oligomerization feed conduit 196 to the oligomerization zone 190 should be kept dry except in an initial start-up phase.

The oligomerization reaction conditions in the oligomerization reactors 192, 210 in the oligomerization zone 190 are set to keep the reactant fluids in the liquid phase. With liquid oligomerate recycle, lower pressures are necessary to maintain liquid phase. Operating pressures include between about 2.1 MPa (300 psia) and about 10.5 MPa (1520 psia), suitably at a pressure between about 2.1 MPa (300 psia) and about 6.9 MPa (1000 psia) and preferably at a pressure between about 2.8 MPa (400 psia) and about 4.1 MPa (600 psia). Lower pressures may be suitable if the reaction is kept in the liquid phase.

For the zeolite catalyst, the temperature in the oligomerization zone 190 expressed in terms of a maximum bed temperature is in a range between about 150° and about 300° C. If diesel oligomerate is desired, the maximum bed temperature should between about 200° and about 250° C. and preferably between about 225° and about 245° C. The weight hourly space velocity should be between about 0.5 and about 5.0 hr⁻¹.

For the SPA catalyst, the oligomerization temperature in the oligomerization reactor zone 190 should be in a range between about 100° and about 250° C. and suitably between about 150° and about 200° C. The weight hourly space velocity should be between about 0.5 and about 5 hr⁻¹.

An oligomerization recovery zone 220 is in downstream communication with the oligomerization zone 190 and the oligomerate conduit 216 which removes the oligomerate stream from the oligomerization zone 190. The oligomerization recovery zone 220 may include one or more fractionation columns for producing the recycle stream in the recycle line 194 which may comprise either a stream comprising C₅ hydrocarbons or a stream comprising C₆⁺ hydrocarbons, a light purge stream in light purge line 222 which may comprise C₄ hydrocarbons, an intermediate purge stream in line 224 which may comprise C₅ hydrocarbons, one or more product streams represented by a gasoline product stream in a gasoline product line 226 and a diesel product stream in a diesel product line 228 and a cracking feed stream represented by cracking feed line 230 all taken from said oligomerate stream in line 216.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated. Additionally, control valves expressed as either open or closed can also be partially opened to allow flow to both alternative lines.

From the foregoing description, one skilled in the art can easily ascertain the essential characteristics of this invention and, without departing from the spirit and scope thereof, can make various changes and modifications of the invention to adapt it to various usages and conditions.

1. A process for recovering a cracked product comprising:
sending a feedstream comprising VGO or a higher boiling hydrocarbon feedstock to a fluid catalytic cracker to provide a cracked product stream;
feeding the cracked product stream to a main fractionation column;
producing an overhead stream from said main fractionation column comprising at least 15 wt-% C₅ hydrocarbons;
taking a depentanizer feed stream from said overhead stream;
feeding said depentanizer feed stream to a depentanizer column;
taking a depropanizer feed stream from said depentanizer column;
feeding said depropanizer feed stream to a depropanizer column;
taking an oligomerization feed stream from said depropanizer column;
feeding said oligomerization feed stream to an oligomerization reactor to produce an oligomerate stream;
taking an FCC recycle stream from said oligomerate stream; and
delivering said FCC recycle stream comprising oligomers to said fluid catalytic cracker.

2-3. (canceled)

4. The process of claim 1 wherein taking a depentanizer feed stream from said overhead stream further comprises compressing a compressor feed stream taken from said overhead stream to provide a compressed stream.

5. The process of claim 4 further comprising separating the compressed stream in a compressor receiver and feeding a compressor receiver bottom stream to a stripping column.

6. The process of claim 5 further comprising stripping said compressor receiver bottom stream in said stripping column to provide said depentanizer feed stream.

7. The process of claim 4 wherein taking a depentanizer feed stream from said overhead stream further comprises separating said overhead stream in an overhead receiver and compressing an overhead receiver stream from said overhead receiver.

8. (canceled)

9. The process of claim 1 further comprising taking a recycle stream from a bottom stream from said depentanizer column and recycling said recycle stream to a primary absorber.

10. The process of claim 1 wherein an overhead stream from said depentanizer is the depropanizer feed.

11. A process for producing hydrocarbons comprising:
feeding a depentanizer feed stream having at least 15 wt-% C₅ hydrocarbons to a depentanizer column;
taking a depropanizer feed stream from said depentanizer column;
feeding said depropanizer feed stream to a depropanizer column to produce an oligomerization feed stream; and
feeding an oligomerization feed stream to an oligomerization reactor to provide an oligomerate stream;
taking a cracking stream from said oligomerate stream;
delivering said cracking stream comprising oligomers to a cracking reactor; and
taking a recycle stream from a bottom stream from said depentanizer column and recycling said recycle stream to a primary absorber.

12. The process of claim 11 further comprising taking said oligomerization feed stream from a bottom of said depropanizer column.

13. The process of claim 11 further comprising taking said depropanizer feed stream from an overhead of said depentanizer column.

14. (canceled)

15. A process for producing hydrocarbons comprising:
compressing a hydrocarbon stream to provide a compressed stream;
taking a depentanizer feed stream from said compressed stream;
feeding said depentanizer feed stream to a depentanizer column;
taking a depropanizer feed stream from said depentanizer column; and
feeding said depropanizer feed stream to a depropanizer column;
taking an oligomerization feed stream from said depropanizer column;
feeding said oligomerization feed stream to an oligomerization reactor to produce an oligomerate stream;
taking a cracking stream from said oligomerate stream;
delivering said cracking stream comprising oligomers to a cracking reactor; and
taking a recycle stream from a bottom stream from said depentanizer column and recycling said recycle stream to a primary absorber.

16-17. (canceled)

18. The process of claim 15 wherein taking a depentanizer feed stream from said compressed stream further comprises separating said compressed stream in a compressor receiver and feeding a compressor receiver bottom stream to a stripping column.

19. The process of claim 18 further comprising stripping said compressor receiver bottom stream in said stripping column to provide said depentanizer feed stream.

20. The process of claim 15 further comprising taking said depropanizer feed stream from an overhead of said depentanizer column.

21. The process of claim 11 further comprising:
sending a feedstream to a fluid catalytic cracker to provide a cracked product stream;
feeding the cracked product stream to a main fractionation column; and
producing an overhead stream from said main fractionation column from which said depentanizer feed stream is taken.

22. The process of claim 21 further comprising compressing a compressor feed stream taken from said overhead stream to provide a compressed stream, separating the compressed stream in a compressor receiver and feeding a compressor receiver bottom stream to a stripping column.

23. The process of claim 22 further comprising stripping said compressor receiver bottom stream in said stripping column to provide said depentanizer feed stream.

24. The process of claim 15 further comprising:
sending a feedstream to a fluid catalytic cracker to provide a cracked product stream;
feeding the cracked product stream to a main fractionation column; and
producing an overhead stream from said main fractionation column as said hydrocarbon stream.

25. The process of claim 24 wherein said overhead stream comprises at least 15 wt-% C₅ hydrocarbons.
